(19) United States
(12) Patent Application Publication
(10) Pub. No.: US 2014/0316448 A1
(43) Pub. Date: Oct. 23, 2014

(54) DEVICES, SYSTEMS AND METHODS FOR A GUIDE WIRE LOADER

(71) Applicant: Cardiovascular Systems, Inc., St. Paul, MN (US)

(72) Inventor: Joseph Higgins, Minnetonka, MN (US)

(73) Assignee: Cardiovascular Systems, Inc., St. Paul, MN (US)

(21) Appl. No.: 14/204,013

(22) Filed: Mar. 11, 2014

Related U.S. Application Data
(60) Provisional application No. 61/781,909, filed on Mar. 14, 2013.

(51) Int. Cl. A61B 17/3207

(52) U.S. Cl. CPC A61B 17/320758; A61B 2017/320733
USPC 606/159; 29/428

(57) ABSTRACT

The present disclosure pertains to a handheld device for loading a guide wire into a medical device. The device has a housing having a slot; a driving wheel disposed within a first portion and rotatably connected to a motor disposed within the housing; a plurality of compression wheels disposed within the second portion of the housing; and a switch disposed on an outer surface of the second portion, the switch in mechanical communication with the compression wheels. When a portion of the guide wire is inserted into the slot and the switch is actuated in a first direction, the compression wheels move toward the driving wheel to grip the guide wire. When the motor is on, the driving wheel rotates to move the guide wire in an axial direction and/or rotational direction.

### CROSS-REFERENCE TO RELATED APPLICATIONS

This application claims priority to App. Ser. No. 61/781,909, entitled “Devices, Systems and Methods for a Guide Wire Loader for Rotational Atherectomy,” filed Mar. 14, 2013.

### FIELD OF THE INVENTION

The present disclosure generally relates to devices and systems for loading guide wires into medical devices, including rotational atherectomy devices. More specifically, a handheld guide wire loader is provided.

### DESCRIPTION OF THE RELATED ART

A variety of techniques and instruments have been developed for use in the removal or repair of tissue in arteries and similar body passageways. A frequent objective of such techniques and instruments is the removal of atherosclerotic plaques in a patient's arteries. Atherosclerosis is characterized by the buildup of fatty deposits (atheromas) in the intimal layer (under the endothelium) of a patient's blood vessels. Very often over time, what initially is deposited as relatively soft, cholesterol-rich atheromatous material hardens into a calcified atherosclerotic plaque. Such atheromas restrict the flow of blood, and therefore often are referred to as stenotic lesions or stenoses, the blocking material being referred to as stenotic material. If left untreated, such stenoses can cause angina, hypertension, myocardial infarction, strokes and the like.

Rotational atherectomy procedures have become a common technique for removing such stenotic material. Such procedures are used most frequently to initiate the opening of calcified lesions in coronary arteries. Most often the rotational atherectomy procedure is not used alone, but is followed by a balloon angioplasty procedure, which, in turn, is very frequently followed by placement of a stent to assist in maintaining patentcy of the opened artery. For non-calcified lesions, balloon angioplasty most often is used alone to open the artery, and stents often are placed to maintain patentcy of the opened artery. Studies have shown, however, that a significant percentage of patients who have undergone balloon angioplasty and had a stent placed in an artery experience stent restenosis—i.e., blockage of the stent which most frequently develops over a period of time as a result of excessive growth of scar tissue within the stent. In such situations an atherectomy procedure is the preferred procedure to remove the excessive scar tissue from the stent (balloon angioplasty being not very effective within the stent), thereby restoring the patentcy of the artery.

Several kinds of rotational atherectomy devices have been developed for attempting to remove stenotic material. In one type of device, such as that shown in U.S. Pat. No. 4,990,134 (Auth), a burr covered with an abrasive abrading material such as diamond particles is carried at the distal end of a flexible drive shaft. The burr is rotated at high speeds (typically, e.g., in the range of about 150,000-190,000 rpm) while it is advanced across the stenosis. As the burr is removing stenotic tissue, however, it blocks blood flow. Once the burr has been advanced across the stenosis, the artery will have been opened to a diameter equal to or only slightly larger than the maximum outer diameter of the burr. Frequently more than one size burr must be utilized to open an artery to the desired diameter.

U.S. Pat. No. 5,314,438 (Shturman) discloses another atherectomy device having a drive shaft with a section of the drive shaft having an enlarged diameter, at least a segment of this enlarged surface being covered with an abrasive material to define an abrasive segment of the drive shaft. When rotated at high speeds, the abrasive segment is capable of removing stenotic tissue from an artery. Though this atherectomy device possesses certain advantages over the Auth device due to its flexibility, it also is capable only of opening an artery to a diameter about equal to the diameter of the enlarged abrading surface of the drive shaft since the device is not eccentric in nature.

U.S. Pat. No. 6,494,890 (Shturman) discloses a known atherectomy device having a drive shaft with an enlarged eccentric section, wherein at least a segment of this enlarged section is covered with an abrasive material. When rotated at high speeds, the abrasive segment is capable of removing stenotic tissue from an artery. The device is capable of opening an artery to a diameter that is larger than the resting diameter of the enlarged eccentric section due, in part, to the orbital rotational motion during high speed operation. Since the enlarged eccentric section comprises drive shaft wires that are not bound together, the enlarged eccentric section of the drive shaft may flex during placement within the stenosis or during high speed operation. This flexion allows for a larger diameter opening during high speed operation, but may also provide less control than desired over the diameter of the artery actually abraded. In addition, some stenotic tissue may block the passageway so completely that the Shturman device cannot be placed therethrough. Since Shturman requires that the enlarged eccentric section of the drive shaft be placed within the stenotic tissue to achieve abrasion, it will be less effective in cases where the enlarged eccentric section is prevented from moving into the stenosis. The disclosure of U.S. Pat. No. 6,494,890 is hereby incorporated by reference in its entirety.

U.S. Pat. No. 5,681,336 (Clement) provides a known eccentric tissue removing burr with a coating of abrasive particles secured to a portion of its outer surface by a suitable binding material. This construction is limited, however because, as Clement explains at CoI. 3, lines 53-55, that the asymmetrical burr is rotated at “lower speeds than are used with high speed ablation devices, to compensate for heat or imbalance.” That is, given both the size and mass of the solid burr, it is infeasible to rotate the burr at the high speeds used during atherectomy procedures, i.e., 20,000-200,000 rpm. Essentially, the center of mass offset from the rotational axis of the drive shaft would result in development of significant centrifugal force, exerting too much pressure on the wall of the artery and creating too much heat and excessively large particles.

Generally atherectomy devices utilize a guidewire that extends distally from the distal end of the drive shaft to assist a practitioner in guiding the device through the patient's vasculature and to a desired location for removal of plaque or fatty tissue buildup. A guidewire, whether a new wire or a replacement wire, must be loaded into the atherectomy device such that it is controllable from a proximal end of the atherectomy device by the practitioner. Prior references that disclose methods and devices for loading a tubular member into a device include U.S. Pat. No. 3,370,150 (Nordgren); U.S. Pat. No. 4,851,694 (Rague); U.S. Pat. No. 5,540,649 (Bonnell); U.S. Pat. No. 5,779,623 (Bonnell); U.S. Pat. No. 6,828,523 (Gysi); U.S. Pat. Pub. No. 2004/0254566 (Plicchi); U.S. Pat. Pub. No. 2006/0161043 (Neumann); U.S. Pat. Pub. No. 2007/0299305 (Murakami); and U.S. 2009/0326449 (Wang), all of which are incorporated herein by reference. These prior art disclosures generally teach in relevant part, devices and systems enabling two-way axial translation of a tubular member using motorized rollers wherein one of the rollers is spring loaded (biased toward the opposite rollers) to increase fit and contact with the tubular member. Additionally, U.S. Pat. Pub. No. 2010/0234873 (Nagano), which is incorporated herein by reference, discloses a drive device for driving a linear body having flexibility, wherein the force applied to drive the linear body only in an axial direction and the pressure applied to a spring is determined and adjusted by a control unit. U.S. Pat. Pub. No. 2012/0232476 (Bhat), which is incorporated herein by reference, discloses a steering system having two radially oppositely arranged driving wheels for steering a tubular member, the drive wheels having a plurality of rollers distributed around a wheel rotation axis of the drive wheels. U.S. Pat. No. 7,955,252 (Suzuki), which is incorporated herein by reference, discloses a treatment tool insertion-retraction and rotating device, the device having a holding member disposed between the treatment tool and a pair of rollers. U.S. Pat. No. 6,786,727 (Irion), which is incorporated herein by reference, discloses a holding device with a fixed gear arrangement with bevel gears to apply a fixed pressure onto an instrument. U.S. Pat. Pub. 2012/0071821 (Yu), which is incorporated herein by reference, discloses a device and method for manipulating an elongated member by actuating rotary members in opposite linear directions to generate rotational motion and actuating rotary members in opposite rotational directions to generate linear motion.

There is a need in the art for a hand-held tool that allows for easy linear and/or rotational movement of a guidewire to be inserted into a medical device.

### BRIEF SUMMARY OF THE INVENTION

The present system is directed in various methods, devices and systems relating to loading a guide wire into a medical device. More specifically, a handheld guide wire loader is provided. The guide wire loader of the present invention allows for entry of the guide wire at any point along its length and is capable of application of axial or axial and torsional/rotational force(s) to the guide wire.

The handheld guide wire loader may comprise a housing having a first portion and a second portion, the first portion and the second portion defining a slot therebetween; a driving wheel disposed within the first portion of the housing, the driving wheel rotatably connected to a motor disposed within the housing; a plurality of compression wheels disposed within the second portion of the housing; and a switch disposed on an outer surface of the second portion, the switch in mechanical communication with the compression wheels. A portion of the guide wire is inserted into the slot, and when the switch is actuated in a first direction, the compression wheels move in the first direction toward the driving wheel to grip the guide wire. When the motor is on, the driving wheel rotates to move the guide wire in an axial direction. In some embodiments, the driving wheel also rotates to move the guide wire in a torsional or rotational direction.

In some embodiments, the handheld guide wire loader may have a plurality of driven wheels within the first portion of the housing. The driven wheels rotatably connected with the driving wheel. In some embodiments, the number of driving wheels and driven wheels in the first portion is greater than the number of compression wheels in the second portion. The center of the compression wheels may in some embodiments be offset from the centers of the driving wheel and driven wheels.

In some embodiments, the driving wheel may have a ramped outer surface, and when the motor is on, the driving wheel rotates to move the guide wire not only axially but also rotationally. The driven wheels may also have a ramped outer surface to further facilitate rotational movement of the guide wire.

A method of loading a guide wire into a medical device, the method comprising: inserting the guide wire into a slot of a handheld guide wire loader, the handheld guide wire loader comprising a housing having a first portion and a second portion, the first portion and the second portion defining a slot therebetween; a driving wheel disposed within the first portion of the housing, the driving wheel rotatably connected to a motor disposed within the housing; a plurality of compression wheels disposed within the second portion of the housing; and a switch disposed on an outer surface of the second portion, the switch in mechanical communication with the compression wheels; actuating the switch in a first direction such that the compression wheels move in the first direction toward the driving wheel to grip the guide wire in the slot; and rotating the driving wheel to move the guide wire in an axial direction. In some embodiments, rotating the driving wheel rotates the guide wire in a rotational direction. In some embodiments, the method further comprises releasing the switch once the guide wire is fully loaded in the medical device, wherein releasing the switch causes the compression wheels move in a second direction opposite the first direction to releasing the guide wire.

### BRIEF DESCRIPTION OF THE DRAWINGS

FIG. 1 shows an embodiment of an atherectomy device with a guide wire.

FIG. 2A is a front cross-sectional view of an embodiment of a guide wire loader device of the invention.

FIG. 2B is a side cross-sectional view of the guide wire loader device of the embodiment shown in FIG. 2A.

FIG. 2C is a front cross-sectional view of a guide wire loader device of the invention with the guidewire inserted.

FIG. 3A is a cross-sectional view of an embodiment of a guide wire loader device of the invention.

FIG. 3B is a side view of an embodiment of the wheels of the embodiment of FIG. 3A.

### DETAILED DESCRIPTION

While the invention is amenable to various modifications and alternative forms, specifics thereof are shown by way of example in the drawings and described in detail herein. It should be understood, however, that the intention is not to limit the invention to the particular embodiments described. On the contrary, the intention is to cover all modifications, equivalents, and alternatives falling within the spirit and scope of the invention.

Various embodiments of the present invention comprise a rotational atherectomy system as described generally in U.S. Pat. No. 6,494,890, entitled “ECCENTRIC ROTATIONAL ATHERECTOMY DEVICE,” which is incorporated herein by reference. Additionally, the disclosure of the following co-owned patents or patent applications are herein incorporated by reference in their entireties: U.S. Pat. No. 6,295,712, entitled “ROTATIONAL ATHERECTOMY DEVICE”; U.S. Pat. No. 6,132,444, entitled “ECCENTRIC DRIVE SHAFT FOR ATHERECTOMY DEVICE AND METHOD FOR MANUFACTURE”; U.S. Pat. No. 6,638,288, entitled “ECCENTRIC DRIVE SHAFT FOR ATHERECTOMY DEVICE AND METHOD FOR MANUFACTURE”; U.S. Pat. No. 5,314,438, entitled “ABRASIVE DRIVE SHAFT DEVICE FOR ROTATIONAL ATHERECTOMY”; U.S. Pat. No. 6,217,595, entitled “ROTATIONAL ATHERECTOMY DEVICE”; U.S. Pat. No. 5,554,163, entitled “ATHERECTOMY DEVICE”; U.S. Pat. No. 7,507,245, entitled “ROTATIONAL ANGIOPLASTY DEVICE WITH ABRASIVE CROWN”; U.S. Pat. No. 6,129,734, entitled “ROTATIONAL ATHERECTOMY DEVICE WITH RADIALLY EXPANDABLE PRIME MOVER COUPLING”; U.S. Pat. No. 8,597,313, entitled “ECCENTRIC ABRADING HEAD FOR HIGH-SPEED ROTATIONAL ATHERECTOMY DEVICES”; U.S. Pat. No. 8,439,937, entitled “SYSTEM, APPARATUS AND METHOD FOR OPENING AN OCCLUDED LESION”; U.S. Pat. Pub. No. 2009/0299392, entitled “ECCENTRIC ABRADING ELEMENT FOR HIGH-SPEED ROTATIONAL ATHERECTOMY DEVICES”; U.S. Pat. Pub. No. 2010/0198239, entitled “MULTI-MATERIAL ABRADING HEAD FOR ATHERECTOMY DEVICES HAVING LATERALLY DISPLACED CENTER OF MASS”; U.S. Pat. Pub. No. 2010/0036402, entitled “ROTATIONAL ATHERECTOMY DEVICE WITH PRE-CURVED DRIVE SHAFT”; U.S. Pat. Pub. No. 2009/0299391, entitled “ECCENTRIC ABRADING AND CUTTING HEAD FOR HIGH-SPEED ROTATIONAL ATHERECTOMY DEVICES”; U.S. Pat. Pub. No. 2010/0100110, entitled “ECCENTRIC ABRADING AND CUTTING HEAD FOR HIGH-SPEED ROTATIONAL ATHERECTOMY DEVICES”; U.S. Design Pat. No. D610258, entitled “ROTATIONAL ATHERECTOMY ABRASIVE CROWN”; U.S. Design Pat. No. D6107102, entitled “ROTATIONAL ATHERECTOMY ABRASIVE CROWN”; U.S. Pat. Pub. No. 2009/0306689, entitled “BIDIRECTIONAL EXPANDABLE HEAD FOR ROTATIONAL ATHERECTOMY DEVICE”; U.S. Pat. Pub. No. 2010/0211088, entitled “ROTATIONAL ATHERECTOMY SEGMENTED ABRADING HEAD AND METHOD TO IMPROVE ABRADING EFFICIENCY”; U.S. Pat. Pub. No. 2013/0018398, entitled “ROTATIONAL ATHERECTOMY DEVICE WITH ELECTRIC MOTOR”; and U.S. Pat. No. 7,666,202, entitled “ORBITAL ATHERECTOMY DEVICE GUIDE WIRE DESIGN.” It is contemplated by this invention that the features of one or more of the embodiments of the present invention may be combined with one or more features of the embodiments of atherectomy devices described therein.

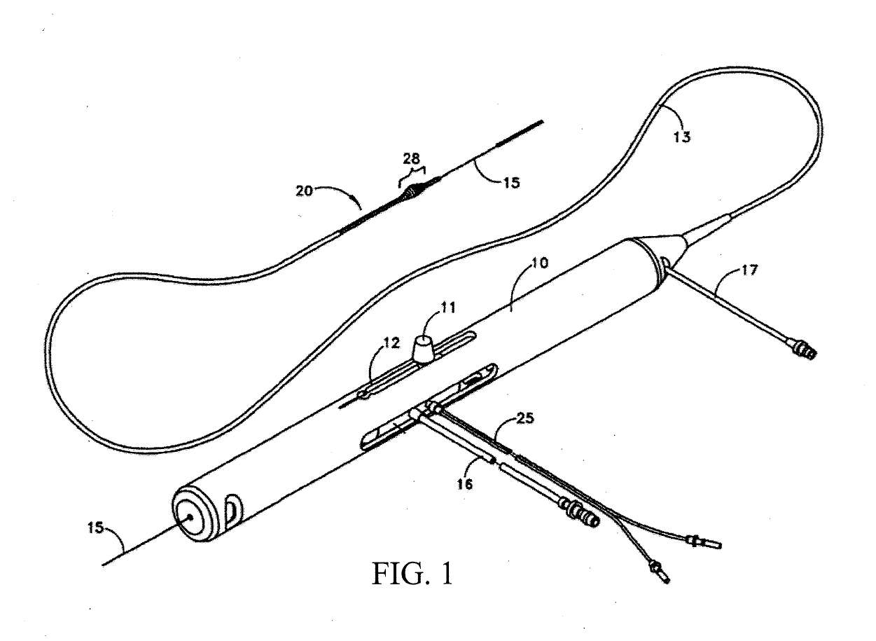

FIG. 1 illustrates an exemplary rotational atherectomy device as described in U.S. Pat. No. 6,494,890, which is incorporated herein by reference. The device includes a handle portion 10; an elongated, flexible drive shaft 20 having an eccentric abrading head 28; and an elongated catheter 13 extending distally from the handle portion 10. The drive shaft 20 is constructed from helically coiled wire as is known in the art and the abrading head 28 is fixedly attached to the drive shaft 20. The drive shaft 20 has an inner lumen, permitting the drive shaft 20 to be advanced and rotated over a guide wire 15. The catheter 13 has a lumen in which most of the length of the drive shaft 20 is disposed, except for the enlarged abrading head 28 and a section of the drive shaft 20 distal to the enlarged abrading head 28. A fluid supply line 17 may be provided for introducing a cooling and lubricating solution (typically saline or another biocompatible fluid) into the catheter 13.

The handle 10 desirably contains a turbine (or similar rotational drive mechanism) for rotating the drive shaft 20 at high speeds. The handle 10 typically may be connected to a power source, such as compressed air delivered through a tube 16. A pair of fiber optic cables 25, alternatively a single fiber optic cable may be used, may also be provided for monitoring the speed of rotation of the turbine and drive shaft 20 (details regarding such handles and associated instrumentation are well known in the industry, and are described, e.g., in U.S. Pat. No. 5,314,407, issued to Auth, and incorporated herein by reference). The handle 10 also desirably includes a control knob 11 for advancing and retracting the turbine and drive shaft 20 with respect to the catheter 13 and the body of the handle.

As discussed above, in at least one embodiment, the eccentric abrading head 28 comprises an eccentric enlarged section of the drive shaft, or an eccentric solid crown, or an eccentric burr attached to the drive shaft. In some embodiments, the abrasive section 28 has a center of mass spaced radially from the rotational axis of the drive shaft 20, facilitating the ability of the device to open the stenotic lesion to a diameter substantially larger than the outer diameter of the abrasive section 28. This may be achieved by spacing the geometric center of the abrasive section 28, i.e., the eccentric enlarged diameter section of the drive shaft 20, or the eccentric solid abrading head or crown, or burr attached to the drive shaft 20, away from the rotational axis of the drive shaft 20. Alternatively, the center of mass of the abrading head 28 may be radially spaced from the drive shaft's rotational axis by providing an abrading head 28 that comprises a differential combination of materials, wherein one side of at least one of the abrading head 28 comprises a more massive or denser material than the other side, which creates eccentricity as defined herein. As those skilled in the art will recognize, creation of eccentricity as by differential use of materials within the structure of the abrading head 28, e.g., a center of mass offset from the drive shaft's rotational axis, is applicable to any embodiment of the abrading head 28 discussed herein, whether concentric, eccentric solid burr, partially hollow crown or abrading head or an enlarged section of the drive shaft, or the equivalent. When rotated at high rotational speeds, the drive shaft 20 stimulates orbital motion of the eccentric abrading head 28 to generate a cutting diameter that is greater than a diameter of the abrading head. In the present invention, the abrading head 28 may comprise a concentric profile or an eccentric profile. In some embodiments, the abrading head 28 may achieve orbital motion, generated by a positioning of the center of mass of the abrading head 28 radially offset from the rotational axis of the drive shaft, either by using different densities of materials and/or geometrically moving the center of mass of the abrading head 28 radially away from the drive shaft's center of mass. This “eccentricity” may be achieved in either a concentric or an eccentric geometric profile. The abrading head 28 may be an enlarged section of the drive shaft, a burr, or a contoured abrasive element and may comprise diamond coating. In other embodiments, the abrading head 28 may comprise a center of mass that is on the drive shaft's rotational axis.

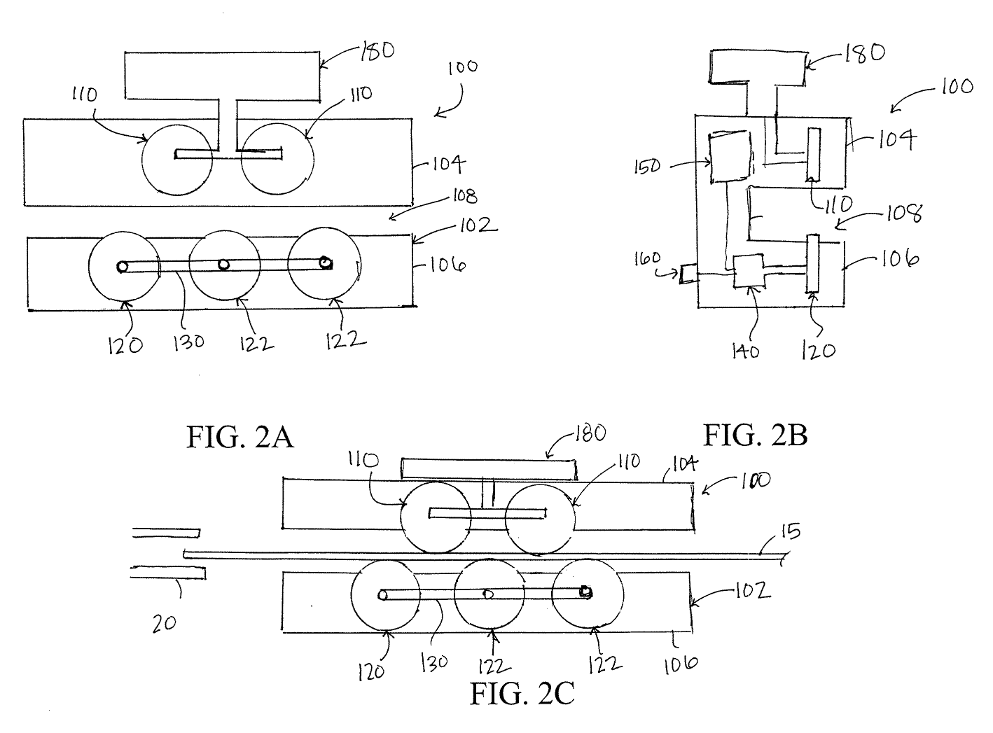

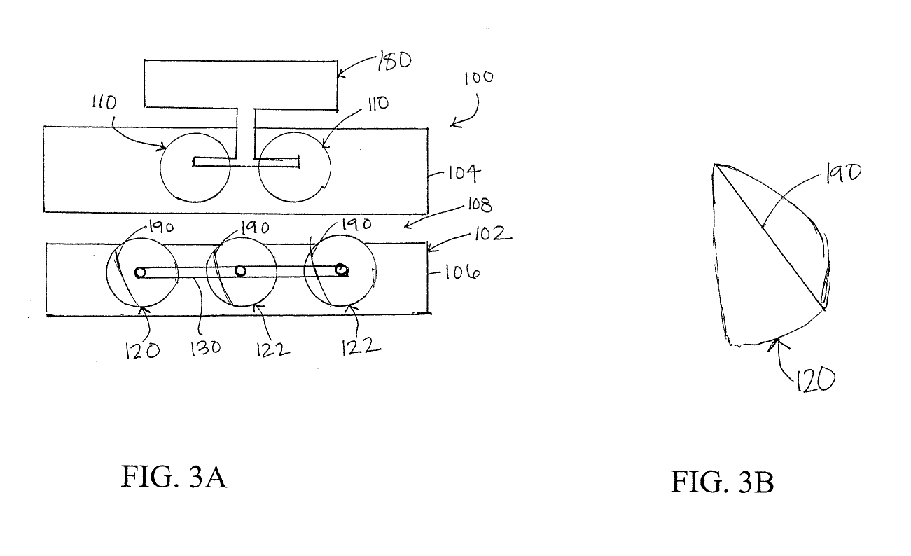

Generally, various embodiments of the present invention comprise a guide wire loading device and system for use with medical devices, including atherectomy devices such as the device described above. Exemplary embodiments of the present invention are illustrated in FIGS. 2A, 2B, 2C and FIGS. 3A, 3B.

FIGS. 2A, 2B, 2C show an embodiment of a guide wire loader device 100, which is a handheld device used to load guide wire 15 into the atherectomy device or other medical device. As shown, the guide wire loader device 100 comprises a housing 102 having an upper portion 104 and a lower portion 106 defining a slot 108 wherein a portion of the guide wire 15 can be inserted for loading into the atherectomy device. Housing 102 may comprise a flexible material. In at least the embodiment shown, a plurality of compression wheels 110 are disposed within the upper portion 104 of the housing 100, and a driving wheel 120 is disposed within the lower portion 106 of the housing 110. In other embodiments contemplated by this invention, the driving wheel may be disposed within the upper portion and the compression wheels are disposed in the lower portion. The compression wheels 110 are disposed on one side of the slot 108 and the driving wheel 120 is disposed on the opposing side of the slot 108, wherein each of the compression wheels 110 and driving wheel 120 are in operable communication with a portion of a guide wire that is engaged within the slot (as shown in FIG. 2C). In at least one embodiment, driving wheel 120 is rotatably connected to additional driven wheels 122 on the same side of the slot 108 as the driving wheel. As shown in FIGS. 2A, 2B, 2C, driven wheels 122 may be connected to driving wheel 120 by a belt 130, such that driving wheel 120 and driven wheels 122 rotate in the same direction. In at least one embodiment, as shown in FIGS. 2A, 2B, 2C, the number of driven wheels 122 is equivalent to the number of compression wheels 110. In at least one embodiment, as shown in FIGS. 2A, 2B, 2C, the number of compression wheels 110 are less than the total number of wheels on the opposite side of the slot, the driving wheel 120 and the driven wheels 122. In some embodiments, the centers of the driving wheel 120 and the driven wheels 122 are offset from the centers of the compression wheels 110, as shown in FIGS. 2A, 2B, 2C. The rotation of driving wheels 120 and driven wheels 122 help propel the guide wire axially through the slot to load the guide wire into the device.

Driving wheel 120 may be driven by a motor 140 rotatably connected to the driving wheel 120. In at least one embodiment, motor 140 is disposed within housing 102. Motor 140 is in electrical communication with a power source 150 such as a battery. In at least one embodiment, power source 150 may be disposed within housing 102, as shown in FIG. 2B. In at least one embodiment, a speed selector 160 may be provided which controls the rotational speed of the motor 140 and thus the rotational speed at which wheels 120, 130 rotate. In addition, an on/off switch may be provided and when the switch is in the “on” position, it operates the motor 140.

Compression wheels 110 may be operably connected to a loading switch 180. In at least the embodiment shown, compression wheels 110 are mechanically connected to a switch 180 disposed on the outer surface of upper portion 104 of the housing 100. When a downward force is applied to the switch 180 (i.e. the switch 180 is engaged), the compression wheels 110 move downwardly into the slot 108 to engage with a guide wire that is inserted into the slot. By engaging the guide wire, the compression wheels 110, along with driving wheel 120 and driven wheels 122, grip the guide wire, and provide a force that helps pull the guide into the device. The switch 180 may also be in electrical communication with the motor, such that the motor turns on when the downward force is applied.

A method for loading a guide wire into a medical device using a guide wire loader such as the one described herein is also provided. A portion of the guide wire 15 is inserted into slot 108. In at least one embodiment, the portion of the guide wire 15 inserted into the slot is between a proximal end and a distal end of the guide wire. In at least one embodiment, the portion of the guide wire inserted into the slot does not initially include either the proximal end or the distal end of the guide wire. The switch is actuated in a first direction such that the compression wheels move in the first direction toward the driving wheel to grip the guide wire in the slot. The driving wheel is rotated to move the guide wire in an axial direction. In some embodiments, rotating the driving wheel rotates the guide wire in a rotational direction. In some embodiments, the method further comprises releasing the switch once the guide wire is fully loaded in the medical device, wherein releasing the switch causes the compression wheels move in a second direction opposite the first direction to releasing the guide wire.

FIGS. 3A-3B illustrate another embodiment of the handheld guide wire loader 100 of the present invention that provides axial movement of the guide wire as in FIGS. 2A-2C, but also rotational movement through torsional force application. In this embodiment, at least the driving wheel 120 and driven wheels 122 have a ramped outer surface 190, as shown in FIG. 3B. Thus, as the compression wheels pull the guide wire axially through the slot, the motorized drag wheel with ramped surface and spring loaded shaft initiate a rotational motion of the guide wire. This combination of axial and rotational movement may be critical in some loading environments.

The present invention should not be considered limited to the particular examples described above, but rather should be understood to cover all aspects of the invention. Various modifications, equivalent processes, as well as numerous structures to which the present invention may be applicable will be readily apparent to those of skill in the art to which the present invention is directed upon review of the present specification.

What is claimed is:

1. A handheld device for loading a guide wire into a medical device, the handheld device comprising:
a housing having a first portion and a second portion, the first portion and the second portion defining a slot therebetween;
a driving wheel disposed within the first portion of the housing, the driving wheel rotatably connected to a motor disposed within the housing;
a plurality of compression wheels disposed within the second portion of the housing; and
a switch disposed on an outer surface of the second portion, the switch in mechanical communication with the compression wheels,
wherein a portion of the guide wire is inserted into the slot, wherein when the switch is actuated in a first direction, the compression wheels move in the first direction toward the driving wheel to grip the guide wire, and wherein when the motor is on, the driving wheel rotates to move the guide wire in an axial direction.

2. The handheld device of claim 1, wherein the driving wheel has a ramped outer surface.

3. The handheld device of claim 2, wherein when the motor is on, the driving wheel rotates to move the guide wire rotationally.

4. The handheld device of claim 1, further comprising a plurality of driven wheels within the first portion of the housing.

5. The handheld device of claim 4, wherein the driven wheels rotatably connected with the driving wheel.

6. The handheld device of claim 4, wherein the center of the compression wheels are offset from the centers of the driving wheel and driven wheels.

7. The handheld device of claim 4, wherein a number of driving wheels and driven wheels in the first portion is greater than a number of compression wheels in the second portion.

8. The handheld device of claim 4, wherein the driven wheels and the driving wheel each have a ramped outer surface.

9. The handheld device of claim 8, wherein when the motor is on, the driven wheels and the driving wheel rotate in the first direction to move the guide wire rotationally.

10. The handheld device of claim 1, wherein the housing comprises a flexible material.

11. The handheld device of claim 1, further comprising a power source disposed within the housing and in electrical communication with the motor.

12. The handheld device of claim 1, wherein the switch is in electrical communication with the motor.

13. The handheld device of claim 1, further comprising a speed selector for controlling a rotational speed of the driving wheel.

14. The handheld device of claim 1, wherein the medical device is an atherectomy device.

15. A method of loading a guide wire into a medical device, the method comprising:
inserting the guide wire into a slot of a handheld guide wire loader, the handheld guide wire loader comprising a housing having a first portion and a second portion, the first portion and the second portion defining a slot therebetween; a driving wheel disposed within the first portion of the housing, the driving wheel rotatably connected to a motor disposed within the housing; a plurality of compression wheels disposed within the second portion of the housing; and a switch disposed on an outer surface of the second portion, the switch in mechanical communication with the compression wheels;
actuating the switch in a first direction such that the compression wheels move in the first direction toward the driving wheel to grip the guide wire in the slot; and
rotating the driving wheel to move the guide wire in an axial direction.

16. The method of claim 15, wherein the axial direction is perpendicular to the first direction.

17. The method of claim 15, wherein rotating the driving wheel rotates the guide wire in a rotational direction.

18. The method of claim 15, wherein the handheld device further comprises a plurality of driven wheels within the first portion of the housing and rotatably connected to the driving wheel.

19. The method of claim 15, wherein the handheld device further comprises a speed selector for selecting the rotational speed of the driving wheel.

20. The method of claim 15, further comprising releasing the switch once the guide wire is fully loaded in the medical device, wherein releasing the switch causes the compression wheels move in a second direction opposite the first direction to releasing the guide wire.
